(19) United States
(12) Patent Application Publication
(10) Pub. No.: US 2016/0222354 A1
(43) Pub. Date: Aug. 4, 2016

(54) Medium for culturing the Marc-145 cell and the preparation method thereof

(71) Applicants: Zhaoqing Dahuanong Biological Medicine Co., Ltd, Zhaoqing (CN); South China Agricultural University, Guangzhou (CN)

(72) Inventors: Ruiai Chen, Zhaoqing (CN); Jiahua Xu, Zhaoqing (CN); Xinqiu Wang, Zhaoqing (CN); Dongxia Zhang, Zhaoqing (CN); Beibei Sun, Zhaoqing (CN); Xiaoyu Xie, Zhaoqing (CN); Changbao Ren, Zhaoqing (CN)

(73) Assignees: Zhaoqing Dahuanong Biological Medicine Co., Ltd; South China Agricultural University

(21) Appl. No.: 14/927,531

(22) Filed: Oct. 30, 2015

(30) Foreign Application Priority Data
Jan. 30, 2015 (CN) 201510050944.4

(51) Int. Cl. C12N 5/071

(52) U.S. Cl. CPC C12N 5/0686; C12N 2500/84; C12N 2500/32; C12N 2500/30

(57) ABSTRACT

The present invention relates to a medium for culturing the M-arc-145 cell, which consists of the following components: the Dulbecco Modified Eagle Medium (DMEM), the solution of gelatin digested by the enzyme and the fetal calf serum; wherein the volume ratio of said DEME, said solution of hydrolytic gelatin by the enzyme and said fetal calf serum is 1 to 2:1 to 5:2 to 5:100. The present invention furthermore provides a preparation method thereof, that is: obtaining the medium by means of taking the solution of gelatin digested by the enzyme, the DEME solution containing the amino acid and the fetal calf serum with the measuring cylinder according to the quantities in the formula, and mixing these components. The medium for culturing the M-arc-145 cell of the present invention may reduce the usage amount of the fetal calf serum during the preparation, hence decreasing the production cost, and of which the preparation method is rather simple and easy to operation.

### CROSS-REFERENCE TO RELATED APPLICATIONS

This application is a continuation in part of, and claims priority to, Chinese Patent Application No. 201510050944.4 with a filing date of Jan. 30, 2015. The content of the aforementioned application, including any intervening amendments thereto, is incorporated herein by reference.

### TECHNICAL FIELD

The present invention relates to a medium for culturing the Marc-145 cell and the preparation method thereof.

### BACKGROUND ART

The Porcine Blue ear disease, also known as the porcine reproductive and respiratory syndrome (PPRS), is a type of infectious disease caused by the porcine reproductive and respiratory syndrome virus, and characterized in the dysgenesis of the adult pigs, premature birth, miscarriage and stillbirth and abnormal breathing of the piglet, and a kind of immunerepress disease, which usually results in others pathogenic infection secondarily. In our country, the prophylaxis to the high pathogenic PRRS is accomplished by means of the attenuated vaccine or the inactivated vaccine. The production course of the attenuated vaccine or the inactivated vaccine against the high pathogenic PRRS includes culturing the Marc-145 cell/hour.

In the existing culturing course of the Marc-145 cell, adding the fetal serum in mass percent of 8 to 10 is usually needed for the culturing, and adding too much less fetal serum may result in the too slow growth and reproduction speed of the Mac-145 cell. For example, the application document of invention patent of which the publication number is CN02002482A has disclosed a production method of the porcine respiratory and productive syndrome virus, where it has disclosed the formula of the cell growth solution, of which the volume percent fraction consists of the DMEM solution from 90% to 92%, the fetal from 8% to 10%, the double-antibody of 1% and the glutamine of 1%; the application document of invention patent of which the publication number is CN101748101A has disclosed a production method of the Porcine respiratory and productive syndrome virus, wherein it has disclosed a growth solution which is the DMEM nutrient solution added the new-born calf serum of 10%. The high price and more volume needed for the culture of the Marc-145 cell has increased the production cost of the vaccine, and increased the bear to the production enterprises and the consumer.

### SUMMARY OF THE INVENTION

The purpose of the present invention is to provide a medium for culturing the Marc-145 cell which needs less fetal serum and gives the high growth and reproduction speed of the Marc-145 cell and the preparation method thereof.

In order to solve the aforesaid problem, the present invention adopts the technical solution as following:

A medium for culturing the Marc-145 cell, which consists of the following components: DMEM, the solution of gelatin digested by the enzyme, the DMEM solution containing the amino acid and the fetal serum; the volume ratio of said DMEM, said solution of gelatin digested by the enzyme, said DMEM solution containing the amino acid and the fetal serum is that said DMEM:said solution of gelatin digested by the enzyme:said DMEM solution containing the amino acid:said fetal serum is equal to 1 to 2:1 to 5:2 to 5:100.

The concentration of the protein in said solution of gelatin digested by the enzyme is from 20 to 80 mg/ml, the solvent in said solution of gelatin digested by the enzyme is the PBS solution;

In said DMEM solution containing amino acid, the solvent is DMEM, and the solvend consists of following components counted in the ration of the mass per unit volume (herein the ration of the mass per unit volume means the ratio of the mass for the solvend components to the volume of the DMEM solution containing amino acid):

5˜35 mg/L of alanine, 10˜60 mg/L of arginine, 15˜80 mg/L of asparagine, 5˜30 mg/L of cystin, 50˜100 mg/L of glutamic acid, 5˜20 mg/L of glycine, 40˜80 mg/L of glutamine, 5˜10 mg/L of histidine, 5˜15 mg/L of isoleucine, 10-20 mg/L of leucine, 10-25 mg/L of lysine, 10-30 mg/L of methionine, 5˜20 mg/L of phenylalanine, 0˜40 mg/L of prolin, 10˜30 mg/L of serine, 10-40 mg/L of threonine, 5-80 mg/L of tryptophan, 10-50 mg/L of tyrosine, 10˜20 mg/L of valine, 5˜10 mg/L of hydroxyproline, 2˜10 mg/L of homocysteine, 5˜15 mg/L of taurine.

In the present invention, a preferred technical solution is that said solution of gelatin digested by enzyme is the solution of gelatin digested by pancreatin, said PBS solution is prepared in the ultrapure water.

In the present invention, a preferred technical solution is that the volume ration of said DMEM, said solution of gelatin digested by enzyme, said DMEM solution containing the amino acid and the fetal serum is that said DMEM:said solution of gelatin digested by enzyme:said DMEM solution containing the amino acid:the fetal serum is equal to 1 to 2:1 to 5:2 to 3:100.

In the present invention, a preferred technical solution is that the volume ration of said DMEM, said solution of gelatin digested by enzyme, said DMEM solution containing the amino acid and the fetal serum is that said DMEM:said solution of gelatin digested by enzyme:said DMEM solution containing the amino acid:the fetal serum is equal to 1 to 2:2 to 5:2 to 5:100.

The solvend in said DMEM solution containing amino acid consists of the following components calculated as the mass per unit volume: 5 mg/L of alanine, 10 mg/L of arginine, 15 mg/L of asparagine, 5 mg/L of cystin, 50 mg/L of glutamic acid, 40 mg/L of glutamine, 5 mg/L of glycine, 5 mg/L of histidine, 5 mg/L of isoleucine, 10 mg/L of leucine, 10 mg/L of lysine, 10 mg/L of methionine, 5 mg/L of phenylalanine, 10 mg/L of prolin, 10 mg/L of serine, 10 mg/L of threonine, 5 mg/L of tryptophan, 10 mg/L of tyrosine, 10 mg/L of valine, 5 mg/L of hydroxyproline, 2 mg/L of homocysteine, 5 mg/L of taurine.

In the present invention, a preferred technical solution is that the volume ration of said DMEM, said solution of gelatin digested by enzyme, said DMEM solution containing the amino acid and the fetal serum is that said DMEM:said solution of gelatin digested by enzyme:said DMEM solution containing the amino acid:the fetal serum is equal to 1:2:2 to 5:100.

The solvend in said DMEM solution containing amino acid consists of the following components calculated as the mass per unit volume: 8 mg/L of alanine, 12 mg/L of arginine, 9 mg/L of asparagine, 20 mg/L of aspartic acid, 7 mg/L of cystin, 60 mg/L of glutamic acid, 50 mg/L of glutamine, 10 mg/L of glycine, 10 mg/L of histidine, 8 mg/L of isoleucine, 12 mg/L of leucine, 12 mg/L of lysine, 15 mg/L of methionine, 6 mg/L of phenylalanine, 10 mg/L of prolin, 10 mg/L of serine, 10 mg/L of threonine, 5 mg/L of tryptophan, 13 mg/L of tyrosine, 10 mg/L of valine, 6 mg/L of hydroxyproline, 5 mg/L of homocysteine, 6 mg/L of taurine.

The present invention also provides a preparation method of the medium for culturing the Marc-145 cell, which including: taking the DMEM, the solution of gelatin digested by enzyme, the DMEM solution containing the amino acid according to the volume ration in the formula and the fetal serum, and mixing them.

In the present invention, a preferred technical solution is that said DMEM solution containing amino acid is prepared according to the following method: taking alanine, arginine, asparagine, aspartic acid, cystine, glutamic acid and glutamine, glycine, histidine, isoleucine, Leucine, lysine, methionine, phenylalanine, proline, serine, threonine, tryptophan and tyrosine, valine, hydroxyl proline, homocysteine, and taurine of the amounts in the formula respectively, and dissolving these components in the DMEM solution of the amount in the formula, thus the DMEM solution containing amino acid may be prepared.

In the present invention, a preferred technical solution is that said solution of gelatin digested by enzyme is prepared by means of the following steps:

a: dissolving the gelatin in the PBS solution, and thus Obtaining the PBS solution containing the gelatin, wherein the concentration expressed by the mass per unit volume of said PBS solution containing the gelatin is 25 to 600 mg/ml;
b: treating PBS solution containing the gelatin by means of performing autoclaved moist heat sterilization, and the temperature is 121° C. and the pressure of said autoclaved moist heat sterilization is 103.4 Kpa;
c: cooling the PBS solution containing the gelatin treated by the step b to the normal temperature, and adding the pancreatin solution in a mass percentage concentration of 0.25%,
(the pancreatin solution of the present invention means the solution prepared by means of adding the pancreatin into the D-Hanks' balance saline solution without Ca),
then enzymlized the gelatin at the temperature of 37° C. for 8 to 24 hours, and repeating the operation of this step three times;
d. cooling the PBS solution containing the gelatin treated by the step c to the temperature of 2 to 8° C., and repeating the step c if the coagulation phenomena occurs, until the coagulation phenomena doesn't occur at the temperature of 2 to 8° C., then the solution of gelatin digested by enzyme is obtained.

In the present invention, the preferred technical solution is that the volume ratio of the pancreatin solution added to the PBS solution containing the gelatin every time is from 1:(9 to 10).

In the present invention, the preferred technical solution is that the enzyme digestion of said step c is for 24 h, and repeating the operations in step c for 2 to 3 time.

Compared to the prior art, the advantage of the present invention lies in that, adding the solution of gelatin digested by enzyme and the DMEM solution containing amino acid may reduce the amount of the fetal serum for culturing the Marc-145 to 2 to 5 percentage of the that in the prior art, which may sharply decrease the amount of the fetal serum used and thus reduce the cost of production; the decrease of the fetal serum reduces the difficulty for the separation caused by the later purification of the virus; furthermore, the preparation method is rather simple, and easy to operate, and is convenient for the need of accomplishing the mass production.

What follows detailed describe the present invention in combination with the specific embodiments.

### SPECIFIC EMBODIMENTS

### Example 1

A medium for culturing the M-arc-145 cell consists of the following components: the DMEM, the solution of gelatin digested by the enzyme, the DEME solution containing the amino acid and the fetal calf serum, said solution gelatin digested by the enzyme and said fetal calf serum is 1:2:X:100; said solution of gelatin digested by the enzyme is the solution of gelatin digested by the pancreatin, said PBS solution is prepared in the ultrapure water.

the concentration of the protein in said solution of gelatin digested by the enzyme is 50 mg/ml, the solvent in said gelatin solution is the phosphate buffer solution (PBS); in said DEME solution containing the amino acid, the solvent is DEME, and the solvend consists of the following components calculated according to the ratio of the mass per unit volume: 5 mg/L of alanine, 10 mg/L of arginine, 5 mg/L of asparagine, 15 mg/L of aspartic acid, 5 mg/L of cystin, 50 mg/L of glutamic acid, 5 mg/L of glycine, 40 mg/L of glutamine, 5 mg/L of histidine, 5 mg/L of isoleucine, 10 mg/L of leucine, 10 mg/L of lysine, 10 mg/L of methionine, 5 mg/L of phenylalanine, 10 mg/L of prolin, 10 mg/L of serine, 10 mg/L of threonine, 5 mg/L of tryptophan, 10 mg/L of tyrosine, 10 mg/L of valine, 5 mg/L of hydroxyproline, 2 mg/L of homocysteine, 5 mg/L of taurine.

The present invention also provides a preparation method for culturing the Marc-145 cells, which comprises taking the solution of gelatin digested by the enzyme, the DEME solution containing the amino acid and the fetal calf serum with the measuring cylinder according to the amounts in the formula, and mixing these components, thus the medium respectively is obtained; said DMEM solution containing amino acids is prepared according to the steps following: obtaining by means of taking the alanine, arginine, asparagine, aspartic acid, cystine, glutamic acid and glutamine, glycine, histidine, isoleucine, leucine, lysine, methionine, phenylalanine, proline, serine, threonine, tryptophan and tyrosine, valine, hydroxy proline, homocysteine, taurine of the amounts in the formula, and then dissolving these components in the DMEM solution of the amount in the formula, thus the said DMEM solution containing amino acid is obtained;

said solution of gelatin digested by the enzyme is prepared by means of the following steps:
a. dissolving 5 g of the gelatin in the PBS solution, and thus Obtaining 90 ml of the PBS solution containing the gelatin;
b. treating PBS solution containing the gelatin by means of performing autoclaved moist heat sterilization, and the temperature is 121° C., the pressure is 103.4 KPa and the sterilization time is 20 min;
c. cooling the PBS solution containing the gelatin treated by the step b to the normal temperature, and adding 10 ml of pancreatin solution in a mass percentage concentration of 0.25%, then digesting the gelatin using the enzyme at the temperature of 37° C. for 24 hours, and repeating the operation of this step for two times;
d. cooling the PBS solution containing the gelatin treated by the step c to the temperature of 2° C. until the coagulation phenomena doesn't occur, then the solution of gelatin digested by enzyme is obtained.

In this example, the mediums in the volume ratio of said solutions of the DEME:said solution of gelatin digested by enzyme:the DMEM solution containing amino acid:said fetal calf serum equal to 1:2:X:100, where the values of X equal to 0.5, 1, 2, 3, 4, or 5 respectively consist the group 2 to group 7, while the mediums containing the same amounts of the others components as the present application but without the fetal serum consists of the group 1; the mediums not adding the solution of gelatin digested by the enzyme and the DMEM solution containing amino acid where the ratios of the added fetal serum to the DMEM solution are 10:100, 8:100, 5:100, 7:100 respectively consist the control group 1 to control group 4; the Marc-145 cells are subcultured in the proportion of 1 to three, and the states of cell culturing are observed 3 h, 1 day, 2 days, and 3 days respectively after inoculation in the course of culturing.

The detailed cases were provided in table 1:

TABLE 1
Growth states table of the Marc-145 cells

| | | |
|---|---|---|
| | Serum | |
| Group | concentration | Observation time |

| | | | | | |
|---|---|---|---|---|---|
| 1 | 0% | − | − | − | − |
| 2 | 0.5% | − | + | ++ | ++ |
| 3 | 1% | + | + | ++ | ++ |
| 4 | 2% | + | ++ | +++ | +++ |
| 5 | 3% | + | ++ | +++ | +++ |
| 6 | 4% | + | ++ | +++ | +++ |
| 7 | 5% | + | ++ | +++ | +++ |
| Control 1 | 10% | + | ++ | +++ | +++ |
| Control 2 | 8% | + | ++ | +++ | +++ |
| Control 3 | 7% | + | ++ | ++ | ++ |
| Control 4 | 5% | − | − | + | ++ |

In the table 1, “−” means that the Marc-145 cells are not at adherence and has stopped growth while “+” means more than 95% of the Marc-145 cell have been at adherence but not in growth, and “++” means the Marc-145 cells present the division and the cells in the division are seldium, and there is not cell in about 50% to 60% of the area of the cell flask; “+++” means that the Marc-145 cells are in good growth state and there is not cell about 20% to 30% of the area in the cell flask; and “++++” means that the Marc-145 cells are in good growth state and there is not cell about 1% to 3% of the area in the cell flask, and the cells have grow to the compact state.

### Example 2

A medium for culturing the M-arc-145 cell consists of the following components: the DMEM, the solution of gelatin digested by the enzyme, the DEME solution containing the amino acid and the fetal calf serum, wherein the volume ratio of said DEME, said solution of gelatin digested by the enzyme and said fetal calf serum is 1:2:Y:100; said solution gelatin digested by the enzyme is the gelatin solution digested by the pancreatin, said PBS solution is prepared in the ultrapure water. The concentration of the protein in said solution of gelatin digested by the enzyme is 70 mg/ml, the solvent in said solution of gelatin digested by the enzyme is the phosphate buffer solution (PBS);

In said DEME solution containing the amino acid, the solvent is DEME, and the solvend consists of the following components calculated according to the ratio of the mass per unit volume: 8 mg/L of alanine, 2 mg/L of arginine, 9 mg/L of asparagine, 20 mg/L of aspartic acid, 7 mg/L of cystin, 60 mg/L of glutamic acid, 50 mg/L of glutamine, 10 mg/L of glycine, 10 mg/L of histidine, 8 mg/L of isoleucine, 12 mg/L of leucine, 12 mg/L of lysine, 15 mg/L of methionine, 6 mg/L of phenylalanine, 10 mg/L of prolin, 10 mg/L of serine, 10 mg/L of threonine, 5 mg/L of tryptophan, 13 mg/L of tyrosine, 10 mg/L of valine, 6 mg/L of hydroxyproline, 5 mg/L of homocysteine, 6 mg/L of taurine.

The present invention also provides a preparation method for culturing the Marc-145 cells, which comprises taking the solution of gelatin digested by the enzyme, the DEME solution containing the amino acid and the fetal calf serum with the measuring cylinder according to the amounts respectively in the formula, and mixing these components, thus the medium is obtained; said DMEM solution containing amino acid is prepared by means of the follow method: obtaining the DMEM solution containing amino acids by means of taking Alanine, arginine, asparagine, aspartic acid, cystine, glutamic acid and glutamine, glycine, histidine, isoleucine, leucine, lysine, methionine, phenylal following method anine, proline, serine, threonine, tryptophan and tyrosine, valine, hydroxy proline, homocysteine, taurine of the amounts in the formula, and then dissolving these components in the DMEM solution of the amount in the formula, thus the DMEM solution containing amino acid is obtained said solution of gelatin digested by the enzyme is prepared by means of the following steps:

a: dissolving 10 g of the gelatin in the PBS solution, and thus Obtaining of the PBS solution containing the gelatin;
b: treating PBS solution containing the gelatin by means of performing autoclaved moist heat sterilization, and the temperature is 121° C., the pressure is 103.4 KPa and the sterilization time is 20 min;
c: cooling the PBS solution containing the gelatin treated by the step b to the normal temperature, and adding 10 ml of the pancreatin solution in a mass percentage concentration of 0.25%, then digesting the gelatin at the temperature of 37° C. for 24 hours, and repeating the operation of this step for three times;
d: cooling the PBS solution containing the gelatin treated by the step c to the temperature of 5° C., and repeating the step c if the coagulation phenomena occur, until the coagulation phenomena stop, thus the solution of gelatin digested by enzyme is obtained.

In this example, the mediums in the volume ratio of said solutions of the DEME:said solution of gelatin digested by enzyme:the DMEM solution containing amino acid:said fetal calf serum equal to 1:2:X:100, where the values of X equal to 0.5, 1, 2, 3, 4, or 5 respectively consist the group 2 to group 7, while the mediums containing the same amounts of the others components as the present application but without the fetal serum consists of the group 1; the mediums not adding the solution of gelatin digested by the enzyme and the DMEM solution containing amino acid where the ratios of the added fetal serum to the DMEM solution are 10:100, 8:100, 5:100, 7:100 respectively consist the control group 1 to control group 4: the Marc-145 cells are subcultured in the proportion of 1 to three, and the state of cell culturing are observed 3 h, 1 day, 2 days, and 3 days after inoculation in the course of culturing. See table 2 for the detailed cases:

TABLE 2
Growth state table of the Marc-145 cell

| | | |
|---|---|---|
| | Serum | |
| Group | concentration | Observation time |

| | | | | | |
|---|---|---|---|---|---|
| 1 | 0% | − | − | − | − |
| 2 | 0.5% | − | + | ++ | ++ |
| 3 | 1% | + | + | ++ | ++ |
| 4 | 2% | + | ++ | +++ | +++ |
| 5 | 3% | + | ++ | +++ | +++ |
| 6 | 4% | + | ++ | +++ | +++ |
| 7 | 5% | + | ++ | +++ | +++ |
| Control 1 | 10% | + | ++ | +++ | +++ |
| Control 2 | 8% | + | ++ | +++ | +++ |
| Control 3 | 6% | − | + | ++ | ++ |
| Control 4 | 5% | − | − | + | ++ |

In the table 2, “−” means that the Marc-145 cells are not at adherence and not in growth while “+” means more than 95% of the Marc-145 cell have been at adherence but not in growth, and “++” means the Marc-145 cells present the division and the cells in the division are less, and there is not cell in about 50% to 60% of the area of the cell flask; “+++” means that the Marc-145 cells are in good growth state and there is not cell about 20% to 30% of the area in the cell flask; and “++++” means that the Marc-145 cells are in good growth state and there is not cell about 1% to 3% of the area in the cell flask, and the cells have grow to the compact state.

### Example 3

A medium for culturing the M-arc-145 cell consists of the following components: the DMEM, the solution of gelatin digested by the enzyme, the DEME solution containing the amino acid and the fetal calf serum, said solution gelatin digested by the enzyme and said fetal calf serum is 1:2:Z:100; the concentration of the protein said solution gelatin digested by the enzyme is 20 mg/ml, the solvent in said gelatin solution is the phosphate buffer solution (PBS); said solution gelatin digested by the enzyme is the solution gelatin digested by the pancreatin, said PBS solution is prepared in the ultrapure water.

In said DEME solution containing the amino acid, the solvent is DEME, and the solvend consists of the following components counted according to the ratio of the mass per unit volume:

80 mg/L of alanine, 60 mg/L of arginine, 25 mg/L of asparagine, 8 mg/ml of aspartic acid 30 mg/L of cystin, 100 mg/L of glutamic acid, 20 mg/L of glycine, 80 mg/L of glutamine, 10 mg/L of histidine, 15 mg/L of isoleucine, 20 mg/L of leucine, 25 mg/L of lysine, 30 mg/L of methionine, 20 mg/L of phenylalanine, 40 mg/L of prolin, 30 mg/L of serine, 40 mg/L of threonine, 80 mg/L of tryptophan,
50 mg/L of tyrosine, 20 mg/L of valine, 10 mg/L of hydroxyproline, 10 mg/L of homocysteine, 15 mg/L of taurine.

The present invention also provides a preparation method for culturing the Marc-145 cells, which comprises taking the solution of gelatin digested by the enzyme, the DEME solution containing the amino acid and the fetal calf serum with the measuring cylinder according to the amounts in the formula, and mixing these components, thus the medium is obtained;

said DMEM solution containing amino acids is prepared according to the steps following: obtaining by means of taking the alanine, arginine, asparagine, aspartic acid, cystine, glutamic acid and glutamine, glycine, histidine, isoleucine, leucine, lysine, methionine, phenylalanine, proline, serine, threonine, tryptophan and tyrosine, valine, hydroxy proline, homocysteine, taurine of the amounts in the formula, and then dissolving these components in the DMEM solution of the amount in the formula, thus the said DMEM solution containing amino acid is obtained;
said solution of gelatin digested by the enzyme is prepared by means of the following steps:
a. Obtaining the PBS solution containing the gelatin by means of dissolving 5 g of the gelatin in 200 ml of PBS solution,
b. treating PBS solution containing the gelatin by means of performing autoclaved moist heat sterilization, and the temperature is 121° C., the pressure is 103.4 KPa and the sterilization time is 20 min;
c. cooling the PBS solution containing the gelatin treated by the step b to the normal temperature, and adding the pancreatin solution in a mass percentage concentration of 0.25%,
then the digesting the gelatin at the temperature of 37° C. for 8 hours, and repeating the operation of this step for three times;
d. cooling the PBS solution containing the gelatin treated by the step c to the temperature of 2° C., and repeating the step c one time if the coagulation phenomena occur, thus the solution of gelatin digested by enzyme is obtained.

In this example, the mediums in the volume ratio of said solutions of the DEME:said solution of gelatin digested by enzyme:the DMEM solution containing amino acid:said fetal calf serum equal to 1:2:X:100, where the values of X equal to 0.5, 1, 2, 3, 4, or 5 respectively consist the group 2 to group 7, while the mediums containing the same amounts of the others components as the present application but without the fetal serum consists of the group 1; the mediums not adding the solution of gelatin digested by the enzyme and the DMEM solution containing amino acid where the ratios of the added fetal serum to the DMEM solution are 10:100, 8:100, 5:100, 7:100 respectively consist the control group 1 to control group 4; the Marc-145 cells are subcultured in the proportion of 1 to three, and the state of cell culturing are observed 3 h, 1 day, 2 days, and 3 days after inoculation in the course of culturing. See table 3 for the detailed cases

TABLE 3
Growth state table of the Marc-145 cell

| | | |
|---|---|---|
| | Serum | |
| Group | concentration | Observation time |

| | | | | | |
|---|---|---|---|---|---|
| 1 | 0% | − | − | − | − |
| 2 | 0.5% | − | + | ++ | ++ |
| 3 | 1% | + | + | ++ | ++ |
| 4 | 2% | + | ++ | +++ | +++ |
| 5 | 3% | + | ++ | +++ | +++ |
| 6 | 4% | + | ++ | +++ | +++ |
| 7 | 5% | + | ++ | +++ | +++ |
| Control 1 | 10% | + | ++ | +++ | +++ |
| Control 2 | 8% | + | ++ | +++ | +++ |
| Control 3 | 6% | − | + | ++ | ++ |
| Control 4 | 5% | − | − | + | ++ |

In the table 1, “−” means that the Marc-145 cells are not at adherence and has stopped growth while “+” means more than 95% of the Marc-145 cell have been at adherence but not in growth, and “++” means the Marc-145 cells present the division and the cells in the division are less, and there is not cell in about 50% to 60% of the area of the cell flask; “+++” means that the Marc-145 cells are in good growth state and there is not cell about 20% to 30% of the area in the cell flask; and “++++” means that the Marc-145 cells are in good growth state and there is not cell about 1% to 3% of the area in the cell flask, and the cells have grow to the compact state.

### Example 4

A medium for culturing the M-arc-145 cell consists of the following components: the DMEM, the solution of gelatin digested by the enzyme, the DEME solution containing the amino acid and the fetal calf serum, said solution gelatin digested by the enzyme and said fetal calf serum is 1:2:M:100; the concentration of the protein in said solution of gelatin digested by the enzyme is 80 mg/ml, the solvent in said gelatin solution is the phosphate buffer solution (PBS); said solution of gelatin digested by the enzyme is the solution of gelatin digested by the pancreatin, said PBS solution is prepared in the ultrapure water.

In said DEME solution containing the amino acid, the solvent is DEME, and the solvend consists of the following components calculated according to the ratio of the mass per unit volume: 20 mg/L of alanine, 35 mg/L of arginine, 20 mg/L of asparagine, 20 mg/L of cystin, 75 mg/L of glutamic acid, 65 mg/L of glutamine, 13 mg/L of glycine, 8 mg/L of histidine, 12 mg/L of isoleucine, 15 mg/L of leucine, 18 mg/L of lysine, 23 mg/L of methionine, 16 mg/L of phenylalanine, 25 mg/L of prolin, 15 mg/L of serine, 30 mg/L of threonine, 70 mg/L of tryptophan, 40 mg/L of tyrosine, 13 mg/L of valine, 9 mg/L of hydroxyproline, 7 mg/L of homocysteine, 10 mg/L of taurine. which comprises taking the solution of gelatin digested by the enzyme, the DEME solution containing the amino acid and the fetal calf serum with the measuring cylinder according to the amounts in the formula, and mixing these components, thus the medium is obtained;

said solution of gelatin digested by the enzyme is prepared by means of the following steps:
a. dissolving 30 g of the gelatin in 200 ml of PBS solution, then Obtaining the PBS solution containing the gelatin
b. treating PBS solution containing the gelatin by means of performing autoclaved moist heat sterilization, and the temperature is 121° C., the pressure is 103.4 KPa and the sterilization time is 20 min;
c. cooling the PBS solution containing the gelatin treated by the step b to the normal temperature, and adding the pancreatin solution in a mass percentage concentration of 0.25%, then digesting the gelatin at the temperature of 37° C. for 16 hours, and repeating the operation of this step for three times;
d. cooling the PBS solution containing the gelatin treated by the step c to the temperature of 5° C. (until the coagulation phenomena doesn't occur), thus the gelatin solution digested by the enzyme is obtained.

The mediums in the volume ratio of said solutions of the DEME:said solution of gelatin digested by enzyme:the DMEM solution containing amino acid:said fetal calf serum equal to 1:2:X:100, where the values of X equal to 0.5, 1, 2, 3, 4, or 5 respectively consist the group 2 to group 7, while the mediums containing the same amounts of the others components as the present application but without the fetal serum consists of the group 1; the mediums not adding the solution of gelatin digested by the enzyme and the DMEM solution containing amino acid where the ratios of the added fetal serum to the DMEM solution are 10:100, 8:100, 5:100, 7:100 respectively consist the control group 1 to control group 4; the Marc-145 cells are subcultured in the proportion of 1 to three, and the state of cell culturing are observed 3 h, 1 day, 2 days, and 3 days after inoculation in the course of culturing. See table 4 for the detailed cases.

TABLE 4
Growth state table of the Marc-145 cell

| | | |
|---|---|---|
| | Serum | |
| Group | concentration | Observation time |

| | | | | | |
|---|---|---|---|---|---|
| 1 | 0% | − | − | − | − |
| 2 | 0.5% | − | + | ++ | ++ |
| 3 | 1% | + | + | ++ | ++ |
| 4 | 2% | + | ++ | +++ | +++ |
| 5 | 3% | + | ++ | +++ | +++ |
| 6 | 4% | + | ++ | +++ | +++ |
| 7 | 5% | + | ++ | +++ | +++ |
| Control 1 | 10% | + | ++ | +++ | +++ |
| Control 2 | 8% | + | ++ | +++ | +++ |
| Control 3 | 6% | − | + | ++ | ++ |
| Control 4 | 5% | − | − | + | ++ |

In the table 4, “−” means that the Marc-145 cells are not at adherence and has stopped growth while “+” means more than 95% of the Marc-145 cell have been at adherence but not in growth, and “++” means the Marc-145 cells present the division and the cells in the division are less, and there is not cell in about 50% to 60% of the area of the cell flask; “+++” means that the Marc-145 cells are in good growth state and there is not cell about 20% to 30% of the area in the cell flask; and “++++” means that the Marc-145 cells are in good growth state and there is not cell about 1% to 3% of the area in the cell flask, and the cells have grow to the compact state.

The above modes of execution are only the preferred modes of execution of the present invention and will not limit the protection scope of the present invention, any unsubstantial changes and alternatives made based on the present invention by the skilled person in the field lie in the protection scope claimed by the present invention.

We claim:

1. A medium for culturing the M-arc-145 cell, characterized in that, it consists of the following components: the dulbecco's modified eagle medium (DMEM), the s solution of gelatin digested by the enzyme, the DEME solution containing amino acid and the fetal calf serum;
wherein the volume ratio of said DEME, said solution of hydrolytic gelatin by the enzyme and said fetal calf serum is that: said DEME:said solution of gelatin digested by the enzyme:said fetal calf serum is equal to 1 to 2:1 to 5:2 to 5:100;
the concentration of the protein in said solution of gelatin digested by the enzyme is 20 mg/ml to 80 mg/ml, the solvent in said gelatin solution is the phosphate buffer solution (PBS);
in said DEME solution containing the amino acid, the solvent is the DEME, and the solvend consists of the following components calculated according to the ratio of the mass per unit volume: 5˜35 mg/L of alanine, 10-60 mg/L of arginine, 15-80 mg/L of asparagine, 5˜30 mg/L of cystin, 50˜100 mg/L of glutamic acid, 5˜20 mg/L of glycine, 40˜80 mg/L of glutamine, 5˜10 mg/L of histidine, 5˜15 mg/L of isoleucine, 10-20 mg/L of leucine, 10-25 mg/L of lysine, 10-30 mg/L of methionine, 5˜20 mg/L of phenylalanine, 10˜40 mg/L of prolin, 10˜30 mg/L of serine, 10-40 mg/L of threonine, 5-80 mg/L of tryptophan, 10˜50 mg/L of tyrosine, 10˜20 mg/L of valine, 5˜10 mg/L of hydroxyproline, 2˜10 mg/L of homocysteine, 5˜15 mg/L of taurine.

2. The medium for culturing the M-arc-145 cell according to claim 1, characterized in that, said solution of gelatin digested by the enzyme is the solution of the gelatin digested by the pancreatin, said PBS solution is prepared with the ultrapure water.

3. The medium for culturing the M-arc-145 cell according to claim 1, characterized in that, the volume ratio of said DEME, said solution of gelatin digested by the enzyme and said fetal calf serum is that: said DEME:said solution of hydrolytic gelatin by the enzyme:said fetal calf serum is equal is equal to 1 to 2:1 to 5:2 to 3:100.

4. The medium for culturing the M-arc-145 cell according to claim 1, characterized in that, the volume ratio of said DEME, said solution of gelatin digested by the enzyme and said fetal calf serum is that: said DEME:said solution of gelatin digested by the enzyme:said fetal calf serum is equal to 1:2:2˜5:100;
the solvend of said DEME containing the amino acid consists of the components according to the following ratio of the mass to the volume: 5 mg/L of alanine, 10 mg/L of arginine, 5 mg/L of asparagine, 5 mg/L of aspartic acid, 5 mg/L of cystin, 50 mg/L of glutamic acid, 40 mg/L of glutamine, 5 mg/L of glycine, 5 mg/L of histidine, 5 mg/L of isoleucine, 10 mg/L of leucine, 10 mg/L of lysine, 10 mg/L of methionine, 5 mg/L of phenylalanine, 10 mg/I of prolin, 10 mg/L of serine, 10 mg/L of threonine, 5 mg/L of tryptophan, 10 mg/L of tyrosine, 10 mg/L of valine, 5 mg/L of hydroxyproline, 2 mg/L of homocysteine, 5 mg/L of taurine.

5. The medium for culturing the M-arc-145 cell according to claim 1, characterized in that, the volume ratio of said DEME, said solution of gelatin digested by the enzyme and said fetal calf serum is equal to 1:2:2 to 5:100; the solvend of said DEME containing the amino acid consists of the components according to the following ration of the mass to the volume: 8 mg/L of alanine, 12 mg/L of arginine, 9 mg/L of asparagine, 20 mg/L of aspartic acid, 7 mg/L of cystin, 60 mg/L of glutamic acid, 50 mg/L of glutamine, 10 mg/L of glycine, 8 mg/L of histidine, 8 mg/L of isoleucine, 12 mg/L of leucine, 12 mg/L of lysine, 15 mg/L of methionine, 6 mg/L of phenylalanine, 10 mg/L of prolin, 10 mg/L of serine, 10 mg/L of threonine, 5 mg/L of tryptophan, 13 mg/L of tyrosine, 10 mg/L of valine, 6 mg/L of hydroxyproline, 5 mg/L of homocysteine, 6 mg/L of taurine.

6. A preparation method of the medium for culturing the M-arc-145 cell according to claim 1, characterized in that, the medium is prepared by means of taking the solution of gelatin digested by the enzyme, the DEME solution containing the amino acid and the fetal calf serum with the measuring cylinder according to the quantities provided by the formula, and mixing these components.

7. The preparation method of the medium for culturing the M-arc-145 cell according to claim 6, characterized in that, said DMEM solution containing amino acid is prepared according to the following steps: obtaining the DMEM solution containing amino acid by means of taking the Alanine, arginine, asparagine, aspartic acid, cystine, glutamic acid and glutamine, glycine, histidine, isoleucine, leucine, lysine, methionine, phenylalanine, proline, serine, threonine, tryptophan and tyrosine, valine, hydroxy proline, homocysteine, taurine in the amounts in the formula, and then dissolving these components in the volume of DMEM solution in the formula,

8. The preparation method of the medium for culturing the M-arc-145 cell according to claim 6, characterized in that, said solution of gelatin digested by the enzyme is prepared by means of the following steps:
a. obtaining the PBS solution containing the gelatin by means of dissolving the gelatin in the PBS solution, wherein the concentration expressed by the mass per unit volume of said the PBS solution containing the gelatin is 25˜600 mg/ml;
b. treating the PBS solution containing the gelatin obtained in step a by performing the autoclaved moist heat sterilization, and where the temperature is 121° C., the pressure is 103.4 KPa and the sterilization time is 20 min;
c. cooling the PBS solution containing the gelatin treated by the step b to the normal temperature, and adding the pancreatin solution at a Mass Percent of 0.25%, then digesting the solution of gelatin by the enzyme at the temperature of 37° C. for 8 to 24 hours, and repeating the operation of this step three times;
d. cooling the PBS solution containing the gelatin treated by the step c to the temperature of 2 to 8° C., and repeating the step c once the coagulation phenomena occurs, until the coagulation phenomena doesn't occurs at the temperature of 2 to 8° C., thus the solution of gelatin digested by the enzyme is obtained.

9. The preparation method of the medium for culturing the M-arc-145 cell according to claim 8, characterized in that, the volume ratio of the pancreatin solution added to the PBS solution containing the gelatin every time in said step c is 1:9 to 10.

10. The preparation method of the medium for culturing the M-arc-145 cell according to claim 8, characterized in that, the time for enzyme digestion in said step c is 24 h, the operation of step c is operated for 2 to 3 repeats.

11. The preparation method of the medium for culturing the M-arc-145 cell according to claim 7, characterized in that, said solution of gelatin digested by the enzyme is prepared by means of the following steps:
a. obtaining the PBS solution containing the gelatin by means of dissolving the gelatin in the PBS solution, wherein the concentration expressed by the mass per unit volume of said the PBS solution containing the gelatin is 25˜600 mg/ml;
b. treating the PBS solution containing the gelatin obtained in step a by performing the autoclaved moist heat sterilization, and where the temperature is 121° C., the pressure is 103.4 KPa and the sterilization time is 20 min;
c. cooling the PBS solution containing the gelatin treated by the step b to the normal temperature, and adding the pancreatin solution at a Mass Percent of 0.25%, then digesting the solution of gelatin by the enzyme at the temperature of 37° C. for 8 to 24 hours, and repeating the operation of this step three times;
d. cooling the PBS solution containing the gelatin treated by the step c to the temperature of 2 to 8° C., and repeating the step c once the coagulation phenomena occurs, until the coagulation phenomena doesn't occurs at the temperature of 2 to 8° C., thus the solution of gelatin digested by the enzyme is obtained.

12. The preparation method of the medium for culturing the M-arc-145 cell according to claim 11, characterized in that, the volume ratio of the pancreatin solution added to the PBS solution containing the gelatin every time in said step c is 1:9 to 10.

13. The preparation method of the medium for culturing the M-arc-145 cell according to claim 11, characterized in that, the time for enzyme digestion in said step c is 24 h, the operation of step c is operated for 2 to 3 repeats.
